(19) United States
(12) Patent Application Publication
(10) Pub. No.: US 2026/0338773 A1
(43) Pub. Date: Oct. 29, 2026

(54) DIAGNOSTIC DEVICE FOR DETECTING PKAL AND VEGF IN PATIENTS WITH RETINAL VASCULATURE DISEASE

(71) Applicant: Alcon Inc., Fribourg (CH)

(72) Inventors: Robert Vincent Brown, Durham, NC (US); David Archer Ellis, Durham, NC (US)

(21) Appl. No.: 19/658,075

(22) Filed: Apr. 24, 2026

Related U.S. Application Data
(60) Provisional application No. 63/794,786, filed on Apr. 25, 2025.

(51) Int. Cl. G01N 33/543; G01N 33/573; G01N 33/58; G01N 33/68

(52) U.S. Cl. CPC G01N 33/54388; G01N 33/573; G01N 33/582; G01N 33/585; G01N 33/6893; G01N 2333/475; G01N 2333/96455; G01N 2800/164

(57) ABSTRACT

Provided herein are point-of-care diagnostic assays and devices for the detection of analytes in liquid ophthalmic samples. The devices herein utilize disposable test strip(s) and a reusable measuring instrument. Detection of major risk factors, including PKal and VEGF, inform more effective decision making for therapeutic intervention of retinal vascular disease.

### RELATED APPLICATIONS

This application claims priority of U.S. Provisional Patent Application No. 63/794,786, filed Apr. 25, 2025, the entire content of which is hereby incorporated by reference.

### BACKGROUND

The current standard of diagnosing retinal vasculature disease (RVD) involves clinical examinations and retinal imaging such as Optical Coherence Tomography (OCT). Retinal imaging methods allow the physician to assess phenotypic indicators of disease stage and severity. Existing diagnostic methods do not provide mechanistic insight into the major modifiable risk factors driving RVD pathogenesis. These factors are highly variable within the RVD patient population.

Major modifiable risk factors in diseases of the retina include vascular endothelial growth factor (VEGF) and plasma kallikrein (PKal). Pkal is a blood product and is only found outside of the circulatory system under abnormal conditions. The presence of Pkal in the aqueous or vitreous humors is abnormal suggesting blood vessel leakage or hemorrhage. [VEGF]>2 ng/mL is abnormal in the aqueous humor or vitreous humor.

Provided herein are diagnostic devices for the detection of PKal, VEGF, or both, in tears (tear film), the aqueous humor, or the vitreous humor of a patient.

### SUMMARY

Disclosed herein are diagnostic assays and devices which provide a simplified point-of-care method for the detection of pathogenic levels of PKal (e.g., [PKal]>0), VEGF (e.g., [VEGF]>2 ng/ml), or both, associated with retinal vascular (or vasculature) disease (RVD) in an ophthalmic sample. The devices herein utilize disposable test strip(s) and a reusable measuring instrument. The disclosed method and device utilize the principle of lateral flow immunochromatography to measure PKal, VEGF, or both in a single assay and determining the amount of PKal, VEGF, or both, or the PKal-VEGF ratio.

In some embodiments, the test strip comprises a single sample application pad containing a sample well which is disposed on a solid support, a conjugate pad having disposed therein a PKal-specific conjugate and a VEGF-specific conjugate, a lateral flow membrane, a PKal-specific test region, a PKal-specific control region, a VEGF-specific test region, a VEGF-specific control region, a reservoir, and a measurement device to determine the concentration of VEGF and the concentration of PKal in the ophthalmic sample. In some embodiments, the ophthalmic sample is a vitreous humor sample. In other embodiments, the ophthalmic sample is an aqueous humor sample. In still other embodiments, the ophthalmic sample is a tear sample.

In some embodiments, the device is a lateral flow immunochromatographic device and the measurement device is a reflectance spectrometer or a fluorometer. In other embodiments, the PKal-specific conjugate and the VEGF specific conjugate may be labeled with a detectable moiety and the detectable moiety may be a microparticle or a dye. In some embodiments, the single sample application pad, the conjugate pad, the PKal-specific test region, the PKal-specific control region, the VEGF-specific test region, the VEGF-specific control region, and the reservoir may all be disposed on a single test strip.

Other embodiments include a method of detecting PKal, VEGF, or both in an ophthalmic sample. In some embodiments, the method includes depositing an ophthalmic sample into a sample application pad of the test cassette described above. In some embodiments of the method, the time for the sample to react with the conjugates and reach an endpoint is from about 2 minutes to about 30 minutes. In another embodiment of the method, the test cassette may be inserted into a measurement device to provide numerical results of the concentration of PKal, VEGF, or both in the sample.

### BRIEF DESCRIPTION OF THE DRAWINGS

FIG. 1 depicts a first view of test strips for use in the disclosed assays and test cassettes.

FIG. 2 depicts a side view of the test strip of FIG. 1.

FIG. 3 depicts a view of a cassette housing the test strip of FIGS. 1 and 2.

FIG. 4 depicts a perspective view of a fluorometer used with the test cassette of FIG. 3.

FIG. 5A depicts a view of a cassette for use in the disclosed assays, showing only detection of PKal.

FIG. 5B depicts a view of a cassette for use in the disclosed assays, showing detection of both PKal and VEGF.

FIG. 6 depicts a graph of levels of VEGF and PKal in DME patients.

### DETAILED DESCRIPTION

### Definitions

Certain terms, whether used alone or as part of a phrase or another term, are defined below.

The articles “a” and “an” refer to one or to more than one of the grammatical object of the article.

Numerical values relating to measurements are subject to measurement errors that place limits on their accuracy. For this reason, the term “about” may explicitly or implicitly modify all numerical values provided herein, unless otherwise indicated.

The term “about” generally indicates a possible variation of a numerical value. In some embodiments, the possible variation (±) may be no more than 10%, 5%, or 1% of the numerical value. In some embodiments, the last decimal place of a numerical value provided herein indicates its degree of accuracy. In some embodiments, where no other error margins are given, the maximum margin is ascertained by applying the rounding-off convention to the last decimal place or last significant digit when a decimal is not present in the given numerical value.

The term “amelioration” means a lessening of severity of at least one indicator of a condition or disease, such as a delay or slowing in the progression of one or more indicators of a condition or disease. The severity of indicators may be determined by subjective or objective measures which are known to those skilled in the art.

The terms “composition” and “pharmaceutical composition” refer to a mixture of at least one compound described herein with another component, such as a carrier or a pharmaceutically acceptable carrier, respectively. The pharmaceutical composition facilitates administration of the compound to a patient or subject.

The terms “effective amount” and “therapeutically effective amount” refer to an amount of therapeutic compound, such as a compound described herein, administered to a subject, either as a single dose or as part of a series of doses, which is effective to produce a desired therapeutic effect.

The term “pharmaceutically acceptable carrier” means a pharmaceutically acceptable material, composition, or carrier, such as a liquid filler, solid filler, stabilizer, dispersing agent, suspending agent, diluent, excipient, thickening agent, solvent, or encapsulating material, involved in carrying or transporting at least one compound described herein within or to the patient such that the compound may perform its intended function. A given carrier must be “acceptable” in the sense of being compatible with the other ingredients of a particular formulation, including the compounds described herein, and not injurious to the patient. Other ingredients that may be included in the pharmaceutical compositions or dosage forms described herein are known in the art and described, for example, in “Remington's Pharmaceutical Sciences” (Genaro (Ed.), Mack Publishing Co., 1985), the entire content of which is incorporated herein by reference.

The term “pharmaceutically acceptable salt” refers to derivatives of the disclosed compounds wherein the parent compound is modified by converting an existing acid or base moiety to its ionized form and paring the ionized parent compound with a counterion to form a salt form. Lists of salts are found in “Handbook of Pharmaceutical Salts: Properties, Selection, and Use” (P. Henrich Stahl & Camille G. Wermuth (Eds.), VHCA & Wiley-VCH, 2002), the entire content of which is incorporated herein by reference.

The term “solid form” includes, but is not limited to, polymorphs, crystalline forms, amorphous forms, solvates, and hydrates of a compound.

The terms “treatment” or “treating” refer to the application of one or more specific procedures used for the amelioration of a disease. A “prophylactic” treatment, refers to reducing the rate of progression of the disease or condition being treated, delaying the onset of that disease or condition, or reducing the severity of its onset.

Recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order, and may include a combination of one or more embodiments herein, unless otherwise indicated herein or otherwise clearly contradicted by context. The use of examples, or exemplary language (“for example,” “such as,” etc.) provided herein is intended merely to better illuminate the described subject matter and does not pose a limitation on the scope of the subject matter otherwise claimed. No language in the specification should be construed as indicating any non-claimed element essential to practicing the described subject matter.

Each group member of a grouping of alternative elements or embodiments of this disclosure may be referred to and claimed individually or in any combination with other members of the group or other elements found herein. Furthermore, a recited member of a group may be included in, or excluded from, another recited group for reasons of convenience or patentability.

Reference made to a patent or printed publication document throughout this specification incorporate herein by reference the document's entire content.

Embodiments of this disclosure are illustrative. Accordingly, the present disclosure is not limited to that precisely as shown and described.

### Plasma Kallikrein and Vascular Endothelial Growth Factor in Retinal Vascular Disease

Disclosed herein is a diagnostic assay and device for the detection of pathogenic levels of major modifiable risk factors associated with retinal vascular (or vasculature) disease (RVD) in a subject or patient, e.g., a subject or patient in need thereof. These risk factors include plasma kallikrein (PKal) and/or vascular endothelial growth factor (VEGF), which may be found in the eyes of patients with RVD. PKal and VEGF (individually or together) may be detected in the aqueous and/or the vitreous humor of patients with RVD. The detection of these proteins may inform more effective decision making for therapeutic intervention.

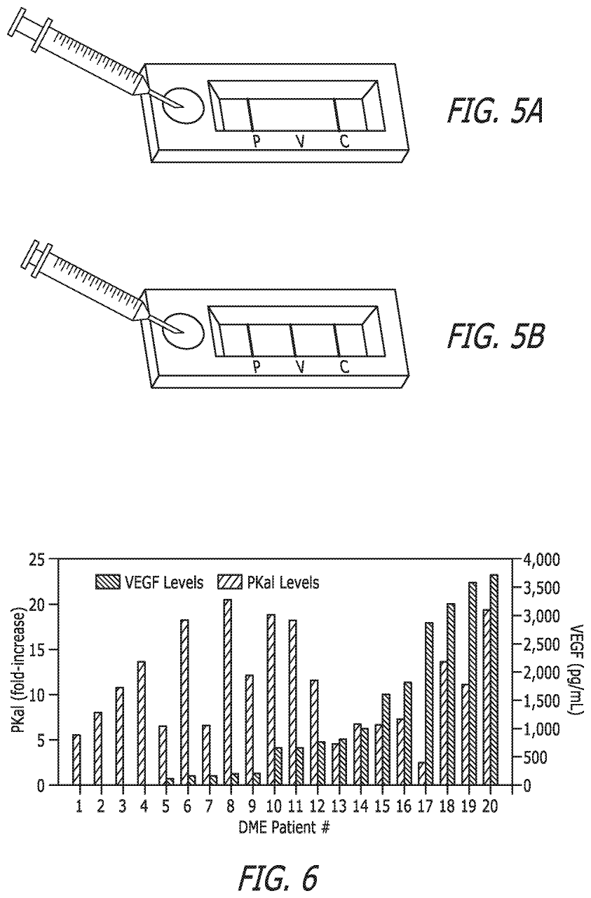

An inevitable consequence of RVD is the extravasation of blood products into the eye from diseased vasculature. PKal is a ubiquitous, endogenous blood-plasma protein that enters the eye from compromised retinal vasculature, causing inflammation and sustained leakage. The role of VEGF-induced angiogenesis in RVD is well established. Abnormally high levels of PKal alone or in concert with elevated levels of VEGF can drive progression of RVD leading to irreversible vision loss (see FIG. 6; A. Kita et al., “Plasma Kallikrein-Kinin System as a VEGF-Independent Mediator of Diabetic Macular Edema” Diabetes. 2015, 64, 3588-3599).

PKal is a trypsin-like serine protease that can liberate kinins from kininogens. It is an essential member of the intrinsic blood coagulation cascade although its role in this cascade does not involve the release of bradykinin or enzymatic cleavage. PKal is encoded by a single gene and synthesized in the liver. It is secreted by hepatocytes as an inactive plasma prekallikrein that circulates in plasma as a heterodimer complex bound to high molecular weight kininogen which is activated to give the active plasma kallikrein. Kinins are potent mediators of inflammation that act through G protein-coupled receptors. Antagonists of kinins (such as bradykinin antagonists) have previously been investigated as potential therapeutic agents for the treatment of several disorders. PKal is thought to play a role in a number of inflammatory disorders. The major inhibitor of plasma kallikrein is the serpin CI esterase inhibitor. Patients who present with a genetic deficiency in CI esterase inhibitor suffer from hereditary angioedema (HAE) which results in intermittent swelling of face, hands, throat, gastro-intestinal tract and genitals. Blisters formed during acute episodes contain high levels of PKal which cleaves high molecular weight kininogen liberating bradykinin leading to increased vascular permeability. Treatment with a large protein PKal inhibitor has been shown to effectively treat HAE by preventing the release of bradykinin which causes increased vascular permeability (A. Lehmann “Ecaliantide (DX-88), a PKal inhibitor for the treatment of hereditary angioedema and the prevention of blood loss in on-pump cardiothoracic surgery” Expert Opin. Biol. Ther. 8, 1187-1199).

The plasma kallikrein-kinin system is abnormally abundant in the vitreous of patients with advanced diabetic macular edema (DME). PKal contributes to retinal vascular dysfunctions in diabetic rats (A. Clermont et al., “Plasma kallikrein mediates retinal vascular dysfunction and induces retinal thickening in diabetic rats” Diabetes. 2011, 60, 1590-1598). Furthermore, administration of the PKal inhibitor ASP-440 ameliorated both retinal vascular permeability and retinal blood flow abnormalities in diabetic rats. Several small-molecule PKal inhibitors are known.

Vascular endothelial growth factor (VEGF) is an important signaling protein involved in both vasculogenesis (the formation of the circulatory system) and angiogenesis (the growth of blood vessels from pre-existing vasculature). The normal function of VEGF is to create new blood vessels during embryonic development, new blood vessels after injury, muscle following exercise, and new vessels (collateral circulation) to bypass blocked vessels. VEGF can contribute to disease. Solid cancers cannot grow beyond a limited size without an adequate blood supply; cancers that can express VEGF are able to grow and metastasize. Overexpression of VEGF can cause vascular disease, including RVD and DME, in the retina of the eye and in other parts of the body. Drugs that inhibit VEGF can control or slow those diseases. Many VEGF inhibitory compounds and known.

In some embodiments, a PKal inhibitor might act as a standalone treatment for RVD or placed in combination with anti-VEGF monotherapy. The latter aspect provides inhibition of PKal to effectively address vascular leakage/inflammation, while anti-VEGF reduces angiogenesis. Informed by a companion diagnostic, physicians would be better equipped to select the optimal therapeutic regimen to address the most relevant modifiable risk factor(s) driving RVD. Importantly, this diagnostic device would guide therapeutic decision making on a patient-by-patient basis.

At present, selection of therapeutic intervention for RVD is an inherently empirical process. Therapies are selected without direct knowledge of specific modifiable risk factors contributing to a patient's underlying RVD-state. Suboptimal responders (up to 50%) to front-line monoclonal antibody therapies are given IVT steroid implants or switched to VABYSMO® (faricimab), a bispecific monoclonal antibody combination therapy.

The ability to diagnostically detect abnormal levels of PKal and/or VEGF in patients with RVD represents an opportunity to introduce personalized medicine to ophthalmology. This technology may provide physicians greater insight into the major modifiable risk factors contributing to RVD. The point-of-care diagnostic may detect the presence of pathologic PKal and/or VEGF in either the aqueous humor or vitreous of a patient. Armed with this insight, physicians can select the appropriate therapy accordingly to treat disease on an individual basis. This method represents an improvement over the current empirical approach.

### Retinal Vascular Disease (RVD)

Common retinal vascular diseases include any variation of retinopathy or retinal edema. Retinopathy is defined as any damage to the retina of an eye, and retinal edema is a condition where fluid builds up in the retina, causing it to swell. The most common types of RVD's include proliferative diabetic retinopathy (PDR), diabetic macular edema (DME), and retinal vein occlusion (RVO). These three disorders account for a large proportion of global severe and moderate vision loss.

### Assays and Devices

The use of diagnostic assays is known for the diagnosis, treatment and management of many diseases. In that regard, different types of diagnostic assays have been developed to simplify the detection of various analytes in clinical samples such as liquid or tissue samples or processed liquid or tissue samples. These assays are frequently expected to provide a fast and reliable result, while being easy to use and inexpensive to manufacture.

One common type of disposable assay device includes a sample addition zone or area for receiving the liquid sample, at least one reagent zone, a reaction zone (also known as a detection zone), and optionally an absorbing zone. These zones can be arranged in order along a fluid passage or channel. These assay devices, commonly known as lateral test strips, can employ a porous material, e.g., nitrocellulose, defining a path for fluid capable of supporting capillary flow. Examples include those devices shown in U.S. Pat. Nos. 5,559,041, 5,714,389, 5,120,643, 6,228,660, 9,128,985, and 9,804,154, U.S. Patent Application No. 2010/0151488, and PCT application Nos. 2016/022655 and 2021/062399, all of which are incorporated herein by reference in their entireties.

The sample addition zone of these assay devices frequently includes a porous material, capable of absorbing the liquid sample, and, when separation of blood cells is required, also effective to trap the red blood cells. Examples of such materials are polymeric membrane filters or fibrous materials, such as paper, fleece, or tissue, comprising e.g., cellulose, wool, glass fiber, asbestos, synthetic fibers, polymers, or mixtures of the same.

The patient's liquid ophthalmic sample is placed in a test cassette that contains reagents to perform the test. As used herein, ophthalmic sample refers to samples taken from tears, the aqueous humor, or the vitreous humor of a patient. In some embodiments, the patient benefitting from the present disclosure includes patients expressing PKal, VEGF, or both above a pathogenic concentration in the patient's liquid ophthalmic sample (e.g., tears, aqueous humor, or vitreous humor). In some embodiments, the ophthalmic sample may be applied directly to the test cassette. Other embodiments include diluting the ophthalmic sample with saline or any other suitable aqueous media prior to application to the test cassette. In some examples, ophthalmic sample may be suspended or treated with suitable reagents and/or media prior to application to the test cassette. The test cassette is then inserted into a test cassette reader that reads, calculates and reports the result.

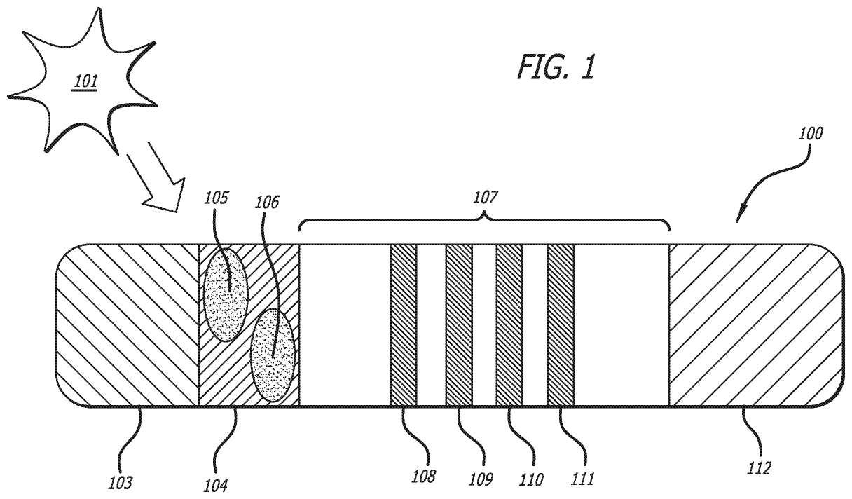

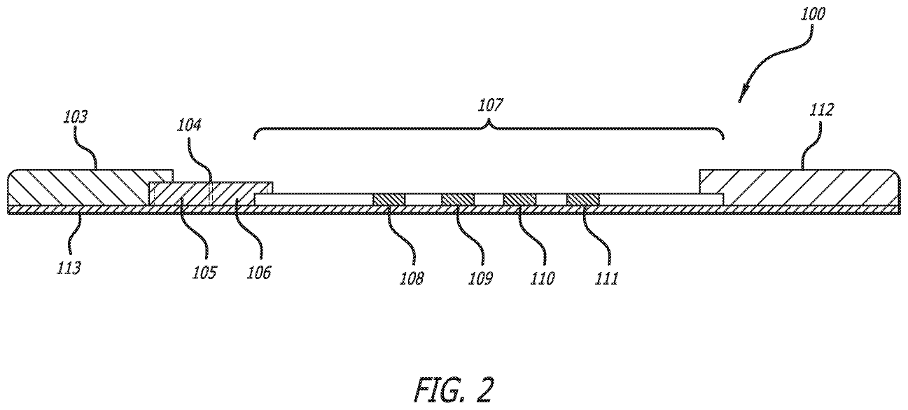

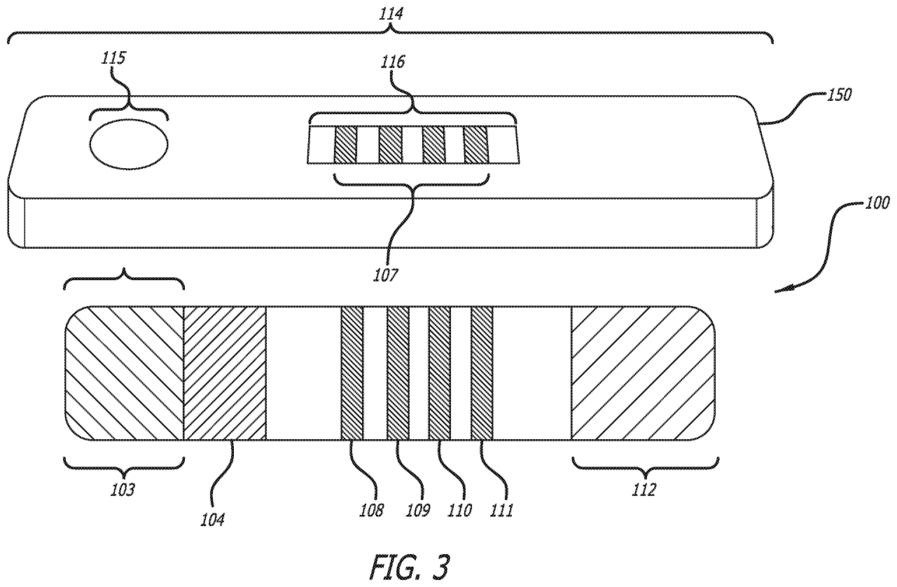

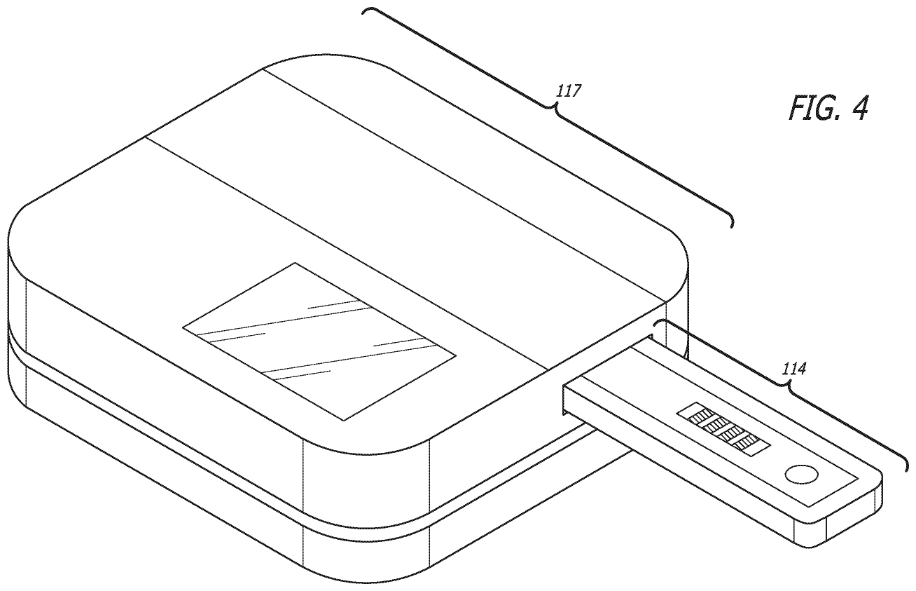

The rapid assay for VEGF, PKal, or both in ophthalmic samples is an immunochromatographic method that utilizes VEGF and PKal binding agents on different test strips or on the same test strip. In order to measure the ratio of VEGF to PKal in ophthalmic samples, two simultaneous measurements are conducted. The first measurement is conducted with a first set of immunochromatographic reagents to measure VEGF. The second measurement is conducted with a second set of immunochromatographic reagents to measure PKal. In one embodiment, both sets of reagents for measuring VEGF and PKal are contained within a single test strip (FIGS. 1 and 2) and a single exterior test cassette (FIG. 3, FIG. 5A, and FIG. 5B) that is inserted into a test cassette reader (FIG. 4) that reads, calculates, and displays the sample assay result. Thus, in some examples, the assay quantifies both VEGF and PKal from a single test strip. In some embodiments, the presence of PKal, VEGF, or both may be detected by visual inspection of a single test strip or of two test strips. In some embodiments, the detection of PKal, VEGF, or both involves binding of the analyte to an analyte binding agent as discussed herein. In other embodiments, detection of PKal, VEGF, or both occurs on the test strip within the test cassette, and the test cassette reader quantifies the label intensity. In some embodiments, the test cassette does not have any mechanical or moving parts. In other embodiments, the user employing the immunochromatographic method described herein adds the ophthalmic sample to the cassette, and the detection and measuring activities can be performed without further manipulation by the user.

One embodiment of a test strip 100 for measuring PKal, VEGF, or both in an ophthalmic sample 101 is shown in FIGS. 1 and 2. The test strip 100 comprises a lateral flow membrane 107, comprised of a material such as, but not limited to, a cellulose nitrate membrane, to which analytical reagents have been fixed to the solid-phase substrate. A sample application pad 103 contacts a conjugate pad 104 containing labeled conjugates comprised of a PKal-specific binding agent and an VEGF-specific binding agent associated with a label. Within conjugate pad 104, conjugates specific for VEGF and PKal are optionally disposed within discrete portions 105, 106, respectively, of conjugate pad 104. The conjugates include a detectable label comprised of a material such as, but not limited to, fluorescent dyes, colored dyes, or microparticles comprised of, in non-limiting examples, a magnetic material, gold, polystyrene, silica, or acrylic. Control regions 109, 111 are provided to bind conjugates that do not react to the sample and are not immobilized at the test regions 108, 110. A reservoir pad 112 is provided at the distal end of the membrane to absorb excess sample fluid and unbound reagents. The test strip is enclosed within a rigid cassette 114 containing a sample well 115 and cassette window 116 to allow for visualization and measurement of the test result.

In some embodiments, the entire assembly of sample pad 103, conjugate pad 104, membrane 107, analytical regions 108, 109, 110, 111, and reservoir pad 112 are held in place with a rigid backing layer 113 with adhesive. In one embodiment, a running buffer is directly dried onto sample application pad 103 and the running buffer is solubilized by the direct application of the ophthalmic sample to the sample application pad without needing to dilute the ophthalmic sample into a running buffer prior to application. In other embodiments, to perform the test, an ophthalmic sample 101 is diluted into a running buffer and is applied to sample pad 103. The buffered ophthalmic sample migrates into the conjugate pad 104 and solubilizes dried conjugates disposed within conjugate pad 104 into solution. In one embodiment, the conjugate pad has disposed therein at least one conjugate specific for PKal and at least one conjugate specific for VEGF. The VEGF and PKal in the ophthalmic sample mix with the conjugates and bind to the respective VEGF-and PKal-specific conjugates. From the conjugate pad, the sample-conjugate complexes pass through membrane 107 containing four analytical regions 108, 109, 110, 111. The four analytical regions 108, 109, 110, 111 contain specific immobilized analyte-binding reagents on membrane 107.

The sample is an ophthalmic sample, typically an ocular fluid (e.g., tears, aqueous humor, or vitreous humor), obtained from an individual patient for whom a PKal concentration, a VEGF concentration, and/or an VEGF-PKal ratio is needed. In some embodiments, the ophthalmic sample comprises a volume of about 1-50 μL. In certain embodiments, the ophthalmic sample volume is about 1-10 μL, about 8-40 μL, about 10-30 μL, about 12-20 μL, or about 1 μL, about 5 μL about 10 μL, about 15 μL, or any volume in a range bounded by any of these values. The ophthalmic sample is diluted into a running buffer at a dilution of sample to running buffer of about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:10, about 1:20, about 1:21, about 1:22, about 1:23, about 1:24, or about 1:25. Suitable running buffers for use in the disclosed systems, methods and kits include buffers comprising a buffered salt solution, a detergent, a protein, and a preservative. In one embodiment, the buffered salt solution is phosphate buffered saline. In another embodiment, the detergent is polysorbate-80. In another embodiment, the protein is casein, or sodium casein. In another embodiment, the preservative is sodium azide. In yet another embodiment, the running buffer is 10 mM phosphate buffer at pH 7.4, 0.15 M NaCl, 0.5% (w/v) polysorbate-80 detergent, 1% (w/v) sodium casein, and 0.02% (w/v) sodium azide.

The PKal test analytical region 108 comprises a first PKal conjugate-specific immobilization reagent, either unbound or bound to a carrier, immobilized on the nitrocellulose membrane. If the ophthalmic sample contains no PKal, unbound PKal-specific conjugates from conjugate pad 104 bind maximally to the PKal test analytical region. If the PKal-specific conjugates have bound to PKal in the ophthalmic sample, there is inhibition of binding of the PKal-specific conjugates to the PKal test analytical region 108, leading to a diminution of the PKal test analytical region signal intensity directly proportional to the concentration of PKal in the ophthalmic sample. The PKal control analytical region 109 captures PKal-specific conjugates, with a second PKal conjugate-specific immobilization retained by the PKal test analytical regardless of whether or not they have bound to ophthalmic PKal. Exemplary carriers for the first PKal conjugate-specific immobilization agent include, but are not limited to, bovine serum albumin (BSA), keyhole limpet hemocyanin (KLH), ovalbumin (OA), dendrimers, and the like. The carrier is an inert molecule which does not cross-react with any of the reagents used for measurement of either analyte or with a non-analyte in the patient sample.

The VEGF test analytical region 110 comprises a first VEGF conjugate-specific immobilization reagent. If the sample contains no VEGF, unbound VEGF-specific conjugates from conjugate pad 104 bind maximally to the VEGF test analytical region. If the VEGF-specific conjugates have bound to VEGF in the ophthalmic sample, there is inhibition of binding of the VEGF-specific conjugates to the VEGF test analytical region 110, leading to a diminution of the VEGF test analytical region signal intensity directly proportional to the concentration of VEGF in the ophthalmic sample. The VEGF control analytical region 111 captures VEGF-specific conjugates, with a second VEGF conjugate-specific immobilization reagent, that have not been retained by the VEGF test analytical region, regardless of whether or not they have bound to ophthalmic VEGF.

The immobilization agent for the test region is a first analyte conjugate-specific reagent such as the analyte (PKal or VEGF) or analyte derivative(s) for a competitive assay, or an analyte conjugate-specific reagent for a sandwich assay. In one embodiment, the test region immobilization agent for PKal is PKal or a PKal derivative such as a PKal-carrier conjugate. In another embodiment, the test region immobilization agent for VEGF is purified human VEGF, recombinant human VEGF, a human VEGF fragment capable of binding the VEGF-specific conjugate, a human VEGF domain capable of binding the VEGF-specific conjugate, or a human VEGF derivative such a human VEGF conjugate comprising of HSA and another protein or molecule which facilitates binding of the HSA to the membrane.

As used herein, the term “derivative” refers to an analyte conjugated to a second molecule which retains at least 95% of its original conformation and binds to an analyte binding agent with similar affinity as the analyte. Exemplary analyte derivatives are analyte conjugated to a carrier protein, or analyte conjugated to a functional group such as biotin.

The immobilization agent for the control region is a second analyte conjugate-specific reagent that binds to the primary labeled anti-analyte conjugate. In one embodiment, the control immobilization agent is a polyclonal antibody specific for the PKal-or VEGF-specific conjugate. In certain embodiments, the control immobilization agent is a polyclonal antibody specific for the species of the PKal or human VEGF-specific conjugate antibody. Non limiting examples of control immobilization agents are goat anti-rabbit antibodies, goat anti-mouse antibodies, goat anti-sheep antibodies, rabbit anti-goat antibodies, rabbit anti-mouse antibodies, rabbit anti-sheep antibodies, mouse anti-goat antibodies, mouse anti-rabbit antibodies, mouse anti-sheep antibodies, sheep anti-goat antibodies, sheep anti-rabbit antibodies, or sheep anti-mouse antibodies.

In one exemplary embodiment, if the PKal-specific conjugate comprises rabbit anti-PKal antibodies and the VEGF-specific conjugate comprises mouse anti-human VEGF, the PKal-specific control immobilization agent is goat anti-rabbit antibodies and the VEGF-specific control immobilization agent is goat anti-mouse antibodies.

The reservoir pad 112 facilitates the uptake of the diluted ophthalmic sample from the test membrane 107 and ensures that capillary action continues to mobilize the conjugates and the buffered ophthalmic sample along the entire length of the membrane and clears the membrane of any unbound conjugates. The reservoir provides additional wicking volume such that the transport of analyte across the test membrane is not solely dependent on the wicking volume of the test membrane 107.

FIG. 3 depicts the entire test cassette 114 which comprises two components, a test strip 100 and an optional rigid case 150. The rigid case of the cassette includes a sample well 115, in which the diluted ophthalmic sample is placed, and a cassette window 116, which provides the reader a view of analytical regions 108, 109, 110, and 111 on the test membrane 107. A structural support 113 is associated with the test strip and is responsible for holding the strip in the correct position and maintenance of contact between the test membrane and other portions of the test strip. The test cassette contains internal structures that make physical contacts with the test strip at various locations to provide sufficient pressure to hold the test strip in place while inside the cassette and to provide sufficient pressure to the overlap regions between test strip components 103, 104, 107, and 112 to keep the components in physical contact. The test cassette can be comprised of any rigid material such as a moldable plastic, a thermoplastic material, or a laminated material.

After a specified length of time, the signal intensity of each of the four analytical regions 108, 109, 110, 111 is quantified with a test cassette reader 117, an exemplary embodiment of which is depicted in FIG. 4. Depending on the type of label associated with the VEGF-specific and PKal-specific conjugates, the test cassette reader 117 has corresponding detection mechanisms to detect and measure the signal intensities from the analytical regions 108, 109, 110, 111.

In one embodiment, the conjugate is labeled with a fluorescent label and the measuring instrument is a fluorometer that quantifies the fluorescence intensity of the four analytical regions 108, 109, 110, 111 on the test strip 101, and to calculate a result from these readings. The intensity of each analytical test regions 108 and 110, is adjusted by dividing each test line intensity with its respective analytical control region 109 or 111. The PKal and VEGF concentrations are determined by comparing the adjusted values to a calibration curve established with pure PKal and VEGF standards and validated with control samples with standardized amounts of both PKal and VEGF. The result is then calculated according to a mathematical algorithm derived from VEGF and PKal standards. The result is expressed as the concentration of ophthalmic VEGF, the concentration of ophthalmic PKal, and the ratio of VEGF to PKal present in the sample.

FIG. 5A and FIG. 5B depict a test cassette which comprises two components, a test strip and an optional rigid case. The rigid case of the cassette includes a sample well, in which an ophthalmic sample (e.g., a diluted ophthalmic sample) is placed, and a cassette window, which provides the reader a view of analytical regions P (PKal), V (VEGF), and C (Control) on the test membrane. A structural support backing can be associated with the test strip and is responsible for holding the strip in the correct position and maintenance of contact between the test membrane and other portions of the test strip. The test cassette contains internal structures that make physical contacts with the test strip at various locations to provide sufficient pressure to hold the test strip in place while inside the cassette and to provide sufficient pressure to the overlap regions between test strip components, including one or more of a sample pad, test membrane, conjugate pad, or reservoir pad, to keep the components in physical contact. The test cassette can be comprised of any rigid material such as a moldable plastic, a thermoplastic material, or a laminated material.

In some embodiments, the length of time required to develop the reaction and obtain a signal intensity of sufficient strength to determine the analyte concentration is from about 2-30 min, about 5 -25 min, about 7-20 min, about 10-17 min, or about 15 min. In other embodiments, the time is less than about 30 min, less than about 25 min, less than about 20 min, less than about 15 min, less than about 10 min, or less than about 5 min. In yet other embodiments, the time is about 5 min, about 6 min, about 7 min, about 8 min, about 9 min, about 10 min, about 11 min, about 12 min, about 13 min, about 14 min, about 15 min, about 16 min, about 17 min, about 18 min, about 19 min, or about 20 min.

In some embodiments, the test cassette reader may be a reflectance spectrophotometer, a fluorometer, or an optical reader, chosen based on the label associated with the analyte-specific conjugate. For example, if the conjugates are labeled with a fluorescent label, the test cassette reader is a fluorometer. If the conjugates are labeled with dyes or colored beads, the test cassette reader is a reflectance spectrophotometer or an optical reader. In certain embodiments, the test cassette can be read by the naked eye if the conjugates are labeled with a dye or colored bead. If the test cassette is read by the naked eye, quantitation of analytes is determined by comparison to colored standards.

In another embodiment, the test cassette reader is a reflectance spectrometer which quantifies a particular wavelength of light reflected from colored microparticles associated with VEGF-specific and PKal-specific conjugates. The amount of reflected light measured at the test band and control band sites is directly proportional to the density of the aggregated microparticles at each site. The data reduction and reporting of the result is as described above for the fluorometer.

In another embodiment, the reader device detects magnetic fields and the labels used in the analyte-specific conjugates are superparamagnetic particles. The data reduction and reporting of the result is as described above for the fluorometer.

### EMBODIMENTS

Embodiment 1. A device for analyzing an ophthalmic sample, comprising:
a test cassette comprising a test strip, wherein the test strip consists essentially of or consists of:
a single sample application pad containing a sample well disposed on a solid support;
a conjugate pad having disposed therein a PKal-specific conjugate and a VEGF-specific conjugate;
a lateral flow membrane;
a PKal-specific test region in which the PKal-specific conjugate which has not been bound by PKal present in the sample is retained by a PKal-specific immobilization agent associated with the membrane and PKal-bound PKal-specific conjugate is not retained by the PKal-specific test region;
a PKal-specific control region in which the PKal-specific bound PKal-specific conjugate is retained by a PKal conjugate-specific immobilization agent associated with the membrane in the PKal-specific control region;
a VEGF-specific test region in which the VEGF-specific conjugate which has not been bound by VEGF present in the sample is retained by a VEGF-specific immobilization agent associated with the membrane and VEGF-bound VEGF-specific conjugate is not retained by the VEGF-specific test region;
a VEGF-specific control region in which the VEGF-bound VEGF-specific conjugate is retained by a VEGF conjugate-specific immobilization agent associated with the membrane in the VEGF-specific control region;
a reservoir; and
a measurement device to determine the concentration of VEGF in the ophthalmic sample and the concentration of PKal in the sample.

Embodiment 2. The device of embodiment 1, wherein the ophthalmic sample is a vitreous humor sample, an aqueous humor sample, or a tear sample of a patient.

Embodiment 3. The device of embodiment 1, wherein the ophthalmic sample is an ocular tissue sample of a patient.

Embodiment 4. The device of embodiment 1, wherein the device is a lateral flow immunochromatographic device.

Embodiment 5. The device of embodiment 1, wherein the measurement device is a reflectance spectrometer or a fluorometer.

Embodiment 6. The device of embodiment 1, wherein the PKal-specific conjugate is an antibody specific for PKal.

Embodiment 7. The device of embodiment 1, wherein the VEGF-specific conjugate is an antibody specific for VEGF.

Embodiment 9. The device of embodiment 1, wherein the PKal conjugate-specific immobilization agent is an antibody specific for the species of the PKal-specific antibody.

Embodiment 10. The device of embodiment 1, wherein the VEGF conjugate-specific immobilization agent is an antibody specific for the species of the VEGF-specific antibody.

Embodiment 11. The device of embodiment 1, wherein the PKal-specific conjugate and the VEGF specific conjugate are labeled with a detectable moiety.

Embodiment 12. The device of embodiment 11, wherein the detectable moiety is a microparticle or a dye.

Embodiment 13. The device of embodiment 12, wherein the microparticles are colloidal gold particles, latex particles, polystyrene particles, acrylic particles or other solid phase microparticles.

Embodiment 14. The device of embodiment 13, wherein the microparticles are colored or tagged with a fluorescent compound.

Embodiment 15. The device of embodiment 1, wherein the single sample application pad, the conjugate pad, the PKal-specific test region, the PKal-specific control region, the VEGF-specific test region, the VEGF-specific control region, and the reservoir are all disposed on a single test strip.

Embodiment 16. A method of detecting PKal and VEGF in an ophthalmic sample comprising:
depositing an ophthalmic sample into a sample application pad of a test cassette according to embodiment 1, wherein the sample passes into the conjugate pad and is exposed to a PKal-specific conjugate and a VEGF-specific conjugate and wherein PKal in the sample binds to the PKal-specific conjugate and VEGF in the sample binds to the VEGF-specific conjugate, wherein the unbound PKal-specific conjugate binds to a PKal-specific immobilization agent in a PKal test region and PKal-bound PKal-specific conjugate passes to a PKal-specific control region which binds the PKal-bound PKal-specific conjugate to the a PKal conjugate-specific immobilization agent, and wherein the unbound VEGF-specific conjugate bind to an VEGF-specific immobilization agent in an VEGF test region and VEGF-bound VEGF-specific conjugate passes to an VEGF-specific control region which binds the VEGF-bound VEGF-specific conjugate to the VEGF conjugate-specific immobilization agent.

Embodiment 17. The method of embodiment 16, wherein the time for the sample to react with the conjugates and reach an endpoint is from about 2 minutes to about 30 minutes.

Embodiment 18. The method of embodiment 17, wherein the time for the sample to react with the conjugates and reach an endpoint is about 10 minutes to about 20 minutes.

Embodiment 19. The method of embodiment 18, wherein the time for the sample to react with the conjugates and reach an endpoint is about 15 minutes.

Embodiment 20. The method of embodiment 16, further comprising inserting the test cassette into a measurement device to provide numerical results of the concentration of PKal and the concentration of VEGF of the sample.

Embodiment 21. A diagnostic method for detecting a concentration of PKal, VEGF, or both in an ophthalmic sample above a pathogenic concentration (e.g., [PKal]>0 or [VEGF]>2 ng/ml, or both), wherein the method employs the device of any one of embodiments 1-15.

Embodiment 22. The diagnostic method of embodiment 21, wherein the ophthalmic sample is a vitreous humor sample, an aqueous humor sample, or a tear sample of a patient.

Embodiment 23. The diagnostic method of embodiment 21, wherein the ophthalmic sample is an ocular tissue sample of a patient.

Embodiment 24. The diagnostic method of one of embodiments 21-23, wherein the ophthalmic sample is an aqueous humor sample or a vitreous humor sample, the pathogenic concentration of PKal is a detectable concentration (e.g., greater than zero), and the pathogenic concentration of VEGF is greater than 2 ng/mL.

### EXAMPLES

The following examples further illustrate embodiments of the present disclosure. However, they are in no way a limitation of the teachings or disclosure as described herein.

### Example 1. Ophthalmic PKal and VEGF Test

To perform the test, an ophthalmic sample is diluted into a running buffer, then the diluted and buffered sample is applied to the sample application pad of a test cassette. The buffered sample migrates into the conjugate pad and solubilizes dried anti-PKal and anti-VEGF antibody conjugates. The PKal and VEGF in the sample mix with the PKal-specific and VEGF-specific conjugates in the conjugate pad, binding to the respective antibody-conjugates. From the conjugate pad, the conjugates pass through the nitrocellulose membrane and over the four analytical regions.

If the sample contains no PKal, the anti-PKal antibody conjugates binds maximally to the PKal test region. If the anti-PKal antibody conjugate has bound to PKal in the sample, there is an inhibition of the anti-PKal conjugate binding to the PKal test analytical region, leading to a reduction of the PKal test analytical region signal intensity proportional to the concentration of PKal in the sample. The PKal control analytical region only binds anti-PKal conjugates that have not bound to the PKal test analytical region, regardless of whether or not they have bound to ophthalmic PKal. After a specified length of time, the signal intensity of each of the PKal test and PKal control analytical regions are quantified with a test cassette reader.

The VEGF test is performed simultaneously on the same test strip as the PKal test. For the VEGF test, an anti-VEGF antibody conjugate is present in the conjugate pad and analytical regions, VEGF test and VEGF control, are included to capture VEGF present in the sample. If the sample contains no VEGF, the anti-VEGF antibody conjugates bind maximally to the VEGF test region. If the anti-VEGF antibody conjugate has bound to VEGF in the sample, there is an inhibition of the anti-VEGF conjugates binding to the VEGF test analytical region, leading to a reduction of the VEGF test analytical region signal intensity proportional to the concentration of VEGF in the sample. The VEGF control analytical region only binds anti-VEGF conjugates that have not bound to the VEGF test analytical region, regardless of whether or not they have bound to ophthalmic VEGF. After a specified length of time, the signal intensity of each of the VEGF test and VEGF control analytical regions are quantified with a test cassette reader.

Unless otherwise defined, all technical and scientific terms used herein are accorded the meaning commonly known to one with ordinary skill in the art.

1. A device for analyzing an ophthalmic sample, comprising:
a test cassette comprising a test strip, wherein the test strip consists essentially of:
a single sample application pad containing a sample well disposed on a solid support;
a conjugate pad having disposed therein a PKal-specific conjugate and a VEGF-specific conjugate;
a lateral flow membrane;
a PKal-specific test region in which the PKal-specific conjugate which has not been bound by PKal present in the sample is retained by a PKal-specific immobilization agent associated with the membrane and PKal-bound PKal-specific conjugate is not retained by the PKal-specific test region;
a PKal-specific control region in which the PKal-specific bound PKal-specific conjugate is retained by a PKal conjugate-specific immobilization agent associated with the membrane in the PKal-specific control region;
a VEGF-specific test region in which the VEGF-specific conjugate which has not been bound by VEGF present in the sample is retained by a VEGF-specific immobilization agent associated with the membrane and VEGF-bound VEGF-specific conjugate is not retained by the VEGF-specific test region;
a VEGF-specific control region in which the VEGF-bound VEGF-specific conjugate is retained by a VEGF conjugate-specific immobilization agent associated with the membrane in the VEGF-specific control region;
a reservoir; and
a measurement device to determine the concentration of VEGF in the ophthalmic sample and the concentration of PKal in the sample.

2. The device of claim 1, wherein the ophthalmic sample is a vitreous humor sample, an aqueous humor sample, or a tear sample of a patient.

3. The device of claim 1, wherein the ophthalmic sample is an ocular tissue sample of a patient.

4. The device of claim 1, wherein the device is a lateral flow immunochromatographic device.

5. The device of claim 1, wherein the measurement device is a reflectance spectrometer or a fluorometer.

6. The device of claim 1, wherein the PKal-specific conjugate is an antibody specific for PKal.

7. The device of claim 1, wherein the VEGF-specific conjugate is an antibody specific for VEGF.

8. The device of claim 1, wherein the PKal conjugate-specific immobilization agent is an antibody specific for the species of the PKal-specific antibody.

9. The device of claim 1, wherein the VEGF conjugate-specific immobilization agent is an antibody specific for the species of the VEGF-specific antibody.

10. The device of claim 1, wherein the PKal-specific conjugate and the VEGF specific conjugate are labeled with a detectable moiety.

11. The device of claim 10, wherein the detectable moiety is a microparticle or a dye.

12. The device of claim 11, wherein the microparticles are colloidal gold particles, latex particles, polystyrene particles, acrylic particles or other solid phase microparticles.

13. The device of claim 12, wherein the microparticles are colored or tagged with a fluorescent compound.

14. The device of claim 1, wherein the single sample application pad, the conjugate pad, the PKal-specific test region, the PKal-specific control region, the VEGF-specific test region, the VEGF-specific control region, and the reservoir are all disposed on a single test strip.

15. A method of detecting PKal and VEGF in an ophthalmic sample comprising: depositing an ophthalmic sample into a sample application pad of a test cassette according to claim 1, wherein the sample passes into the conjugate pad and is exposed to a PKal-specific conjugate and a VEGF-specific conjugate and wherein PKal in the sample binds to the PKal-specific conjugate and VEGF in the sample binds to the VEGF-specific conjugate, wherein the unbound PKal-specific conjugate binds to a PKal-specific immobilization agent in a PKal test region and PKal-bound PKal-specific conjugate passes to a PKal-specific control region which binds the PKal-bound PKal-specific conjugate to the a PKal conjugate-specific immobilization agent, and wherein the unbound VEGF-specific conjugate bind to an VEGF-specific immobilization agent in an VEGF test region and VEGF-bound VEGF-specific conjugate passes to an VEGF-specific control region which binds the VEGF-bound VEGF-specific conjugate to the VEGF conjugate-specific immobilization agent.

16. The method of claim 15, wherein the time for the sample to react with the conjugates and reach an endpoint is from about 2 minutes to about 30 minutes.

17. The method of claim 16, wherein the time for the sample to react with the conjugates and reach an endpoint is about 10 minutes to about 20 minutes.

18. The method of claim 17, wherein the time for the sample to react with the conjugates and reach an endpoint is about 15 minutes.

19. The method of claim 15, further comprising inserting the test cassette into a measurement device to provide numerical results of the concentration of PKal and the concentration of VEGF of the sample.

20. A diagnostic method for detecting a concentration of PKal, VEGF, or both in an ophthalmic sample above a pathogenic concentration, wherein the method employs the device of claim 1.

21. The diagnostic method of claim 20, wherein the ophthalmic sample is a vitreous humor sample, an aqueous humor sample, or a tear sample of a patient.

22. The diagnostic method of claim 20, wherein the ophthalmic sample is an ocular tissue sample of a patient.

23. The diagnostic method of one of claim 20, wherein the ophthalmic sample is an aqueous humor sample or a vitreous humor sample, the pathogenic concentration of PKal is a detectable concentration, and the pathogenic concentration of VEGF is greater than 2 ng/mL.
